(19) United States
(12) Patent Application Publication
(10) Pub. No.: US 2009/0035386 A1
(43) Pub. Date: Feb. 5, 2009

(54) CONDITIONING OF A PATIENT'S BLOOD BY GASES

(76) Inventors: Georg MATHEIS, Burladingen (DE); Andreas Maurer, Tuebingen (DE); Gareth Roberts, Great Shelford (GB)

Correspondence Address: HARNESS, DICKEY & PIERCE, P.L.C., P.O. BOX 828, BLOOMFIELD HILLS, MI, 48303, US

(21) Appl. No.: 12/180,868

(22) Filed: Jul. 28, 2008

(30) Foreign Application Priority Data
Jul. 31, 2007 (DE) 10 2007 038 121.4

(51) Int. Cl. A61K 33/00; A61P 7/00

(52) U.S. Cl. 424/613; 424/700; 424/600

(57) ABSTRACT

The present invention relates to a method for using gas exchange modules for adjusting a pH value of blood in order to, e.g., adjust a non-physiological pH in patients treated with drugs whose activity optimum is in a non-physiological pH or to bring the pH to a physiological value.

### CROSS REFERENCES TO RELATED APPLICATIONS

This application claims priority from German patent application DE 10 2007 038 121.4, filed on Jul. 31, 2007, the contents of which are incorporated herein by reference.

### BACKGROUND OF THE INVENTION

The present invention relates generally to the support of regenerative therapies for injured tissues/organs in a patient's body, and to the support of pharmacotherapies or gene therapies in a patient. This includes blood circulations inside and outside the body, organs perfused separately from the organism, and the treatment of removed blood.

Many pathological processes, injuries and infections cause danger to, damage to or even the death of tissues and/or organs in the human body.

Whereas it is often possible to treat the direct clinical hazards derived therefrom, or to stabilize the conditions, long-term damage or impairments remain with the patients, with which they in the worst case have to cope life-long.

Medical regenerative therapies are based on cellular approaches in which cells or other factors are introduced into the patients in order to stimulate repair mechanisms and the growth of new cells, whereby the intention is to treat the problems of the tissues/organs which have been caused by the intervention, or the damage or impairment.

It has been possible to show in scientific studies that the body responds during the damage process with a locally restricted or systemic inflammatory reaction. This inflammatory reaction can stimulate the body's own repair systems and can also be utilized as stimulus for regenerative medical therapies. However, it has also been shown that large or excessive inflammatory reactions may damage tissues or organs, and may also have a negative influence on the efficacy of medical regenerative therapies, or may even lead to their failure.

It has moreover now been found that the pH in the body plays a large role in the functionality of cells/tissues. The pH is not the same in all regions of the body, but its value in the respective organs/tissues is of crucial importance since only then is it possible for the chemical reactions to proceed under ideal conditions in the respective organ. The pH has effects inter alia on the structure of cell constituents, the permeability of cell walls and the synthesis and breakdown of proteins. It is also important for the activity of hormones and enzymes and the distribution of electrolytes. The pH is particularly important for blood, where pH variations scarcely occur in healthy people. The pH of the blood of a healthy person is between pH 7.36 and pH 7.45. All metabolic reactions are pH-dependent and can proceed optimally only within this range.

In this connection, for example Brooks et al., “Modulation of VEGF production by pH and glucose in retinal Muller cells”, Curr. Eye Res., 1998, 17:875-882, showed that the production of vascular endothelial growth factor (VEGF) in Muller cells of the retina could be increased by raising the pH and raising the glucose, whereas it was possible to reduce VEGF production with a decrease in the pH and a decrease in glucose. This research group concluded in connection with their results that when hypoxia plus acidosis and hypoglycemia exist, as occurs in severe tissue ischemia, glial cells are no longer able to upregulate VEGF synthesis, whereas alkalosis or hyperglycemia may augment hypoxia-induced VEGF production.

It has further been shown with many pharmacotherapies that drugs show a different effect at different pH values in a patient's body or in particular organs or tissues. In extreme cases, the effect may be completely lost owing to incorrect pH. This shows that a pharmacological approach is often successful only under particular physiological conditions.

Thus, for example, Kinoshita et al., “Mild alkalinization and acidification differentially modify the effects of lidocaine or mexiletine on vasorelaxation mediated by ATP-sensitive K+ channels”, Anesthesiology, 2001; 95:200-201, showed that a change in the pH in the rat aorta leads to different effects of the drug lidocaine on the decrease in vascular tension.

Achike and Dai, “Influence of pH changes on the actions of Verapamil on cardiac excitation-contraction coupling”, Eur. J. Pharmacol., 1991, 196:77-83, showed that the effect of verapamil as calcium antagonist was increased during acidosis or alkalosis in rat cardiac cells stimulated with adrenaline or potassium, from which it was concluded that acidosis or alkalosis inhibit the potassium-stimulated contractions of the heart and thus enhance the effect of verapamil.

There is thus a great need inter alia in regenerative medicine and in pharmacotherapy to support regeneration of the injured or damaged tissue, or the effect of a drug, in order to make successful healing of the affected tissues/organs possible and make effective treatment of the patient with drugs possible.

### SUMMARY OF THE INVENTION

It is therefore an object of the present invention to provide aids with which a regenerative therapy in a patient's body and the use of drugs, is supported and promoted by a targeted adjustment of the pH in a simple manner and without major interventions which cause additional stress for the patient's body.

This object is achieved according to the invention by a method for adjusting the pH value of blood, e.g. of a patient's blood, and/or of blood in an isolated organ comprising the steps of:
establishing a connection between said blood and a gas exchange module; and
supplying gas via the gas exchange module to the blood.
   This can take place in blood circulations outside the body or on isolated blood.
This object is further achieved by a method for adjusting a pH value of blood, wherein the method includes the step of employing a gas exchange module.

The object underlying the invention is completely achieved thereby.

It is possible by using gas exchange modules to adjust the pH of blood in a patient's organ rapidly and easily. The patient's body is advantageously in this way not exposed to additional pH-influencing substances.

“Gas exchange module” shall—in the present case—mean any apparatus with which gases can be supplied to and/or removed from, or exchanged in, a patient's blood on connection to the apparatus, especially oxygen, carbon dioxide and nitrogen. Thus, for example, oxygenators which are employed in connection with heart-lung machines are covered in the present case by the term “gas exchange module”. Oxygenators are used to enrich blood with oxygen and remove carbon dioxide from the blood; these are employed for example for respiratory failure or in heart surgery.

The gas exchange module is in this connection for example connected to a patient's blood system via appropriate accesses such as, for example, by placing a needle in the desired blood vessels, and the blood is gassed in the gas exchange module. The module is connected during this via an inlet to a gas supply, and has a gas outlet. The pH of the blood can be adjusted in a targeted and controllable manner by gassing with gases such as CO₂, oxygen, nitrogen or mixtures thereof, because the amount and the nature of the gas employed can be determined specifically for the particular use. Use is made in this connection of the fact that the pH of the plasma and the erythrocytes, the most important constituents of blood, can be influenced by various factors such as, for example, via the concentration of carbon dioxide (CO₂): if the CO₂ concentration falls, the pH rises. In normal circumstances, i.e. in a healthy person, the pH of blood is kept constant by a buffer system.

Thus, it is possible for example in patients with chronic acidosis or alkalosis to change the pH to physiological values. Acidosis or alkalosis can in this connection be detected for example by a sensor in the blood stream, which controls the gas stream and thus the physiological pH.

On the other hand, it is possible with patients receiving a therapy with medicaments whose activity optimum is outside the physiological pH for the pH to be changed, through use of the gas exchange module, to non-physiologically acidic or basic values at which the respective therapy has a better outcome.

The use of gas exchange modules additionally has the great advantage that for example targeted adjustment of the pH of individual organs which are perfused in isolation is possible, so that the pH of the blood in the unperfused organs and extremities remains unaffected. It is possible in this way for the pH in the patient's body to be controlled easily and in a stress-free manner, it being possible to adjust the pH locally.

It is therefore preferred in one embodiment of the use according to the invention for the gas exchange module to be used to adjust the pH of a patient's blood in a target area which is perfused in isolation.

“Perfused separately/in isolation” means a method in which an artificial circulation which is isolated from the blood circulation of the body, which is also called the body's circulation hereinafter, is established and maintained in a target area, i.e. for example an organ, of a human or animal body.

This so-called isolated perfusion of organs or body regions has been used for a long time in order to administer highly active medicaments in the target area and at the same time avoid their side effects on the remainder of the organism, or to employ medicaments in such high concentrations that, on general application to the whole body, unacceptably severe side effects and intolerances would occur.

In the context of the present invention “target area” shall mean an organ which can be isolated in terms of the blood circulation from the rest of the body, or a body region which can be isolated, such as, for example, extremities, i.e. arm or leg, and pelvis.

The size of the module to be employed depends in this connection on the respective use, i.e. whether the gas exchange module is to be employed for adjusting the pH of the total blood volume of a patient or only for a target area/organ perfused separately. Thus, for example, a gas exchange module useful for organs perfused in isolation enables a volumetric flow of blood of 0.1-1.5 l/min and provides a gas exchange area of about 0.01 to 1 m². A module which can be employed for adjusting the pH of the total blood volume of a patient may have for example from 0.5l/min to 7 l/min and provides a gas exchange area of 0.1 to 3 m².

According to one aspect of the invention, the gas exchange module has a gassing membrane, preferably a flat membrane, a hollow fiber membrane or a microfluidic system.

With gassing membranes, or on use of gassing membranes for targeted adjustment of the pH of blood, the gas side is separated from the blood side by a gas-permeable membrane—in a similar way to the human lung. The gas exchange therefore takes place along the gas-permeable membrane owing to a partial pressure difference of the gases employed. The gas is supplied to the module through a gas inlet in the hollow fibers, or on one side of the flat membrane; the blood flows outside the hollow fibers, or on the other side of the flat membrane, during which the gas exchange takes place.

Hollow fiber membranes have the advantage that they have a very much higher surface area (per unit volume) than flat membranes, making it possible for gas exchange modules with hollow fiber membranes to be smaller in size for the same gas exchange capacity than gas exchange modules with flat membranes.

In hollow fiber membranes, the blood flows outside the hollow fibers, while a flushing gas (air, oxygen, CO₂ or other gas mixtures) flows through the inside of the fibers. Between blood and gas, owing to a concentration gradient, there is exchange of the gases at the membrane, such as, for example, of oxygen and carbon dioxide. The principle is the same with flat membranes.

The gassing membranes may in this connection include a material which is selected from polypropylene (PP), polymethylpentene (PMP), silicone, silicone-coated, or other gas-permeable membrane materials. These materials are already employed successfully and tested in connection with gas exchange modules in the state of the art.

The hollow fiber membranes may moreover be disposed in the gas exchange module for example as bundles, stacked mats or rolled mats, it being possible for the distance of the hollow fibers from one another to be adapted to the use desired in each case, taking account of the fact that the distance of the fibers from one another influences the blood resistance and flow rates of the system. It is further possible to provide for use of a pump on the blood side. The use of a pump is, however, not absolutely necessary because a gassing membrane with low flow resistance can be employed for example for patients with good hemodynamic conditions, and the blood is passed over the membrane, and can be enriched with gases there, merely through the arteriovenous pressure difference.

According to another aspect of the invention, the gas exchange module is an artificial lung or an oxygenator.

“Artificial lung” means in the present case any apparatus which takes over the function of the lung temporarily or permanently.

Thus, for example, the iLA membrane ventilator of the applicant (see, e.g., www.novalung.de) can be employed. The iLA membrane ventilator is normally employed for ventilation outside the lung in the case of respiratory failure. The harmful influences of mechanical ventilation can be reduced or even avoided by the iLA membrane ventilator, thus avoiding the risk of overdistension of the lungs and the further damage to the lungs and other organs associated therewith.

The iLA Membrane Ventilator® is an enabling device for advanced protective ventilation. Gas exchange is performed by a heparin coated, biocompatible diffusion membrane. The iLA Membrane Ventilator® is connected to the patient via arteria and venous femoral cannulae. Typical cannula sizes are usually 13 or 15 F arterial and 15 or 17 F venous. Vascular access is achieved via Seldinger's technique.

It is now possible to supply through the iLA membrane ventilator a gas such as, for example, CO₂, oxygen, or nitrogen, to the blood, with the gas exchange taking place at the membrane provided in the iLA, and the blood being enriched with the appropriately supplied gas.

“Oxygenator” means in the present case any medical apparatus with which oxygen and carbon dioxide in the blood of a patient can be exchanged during surgical interventions where the blood stream in the body must be interrupted or stopped for surgical reasons. The oxygenator can moreover be employed for example in connection with heart-lung machines, or else in the extracorporeal oxygenation of blood.

Examples of oxygenators which can be employed for the use according to the invention are oxygenators supplied by Medtronic Inc. USA, Maquet Cardiopulmonary, Germany, Cobe CV, USA, Sorin Biomedica, Italy. An oxygenator supplies vital oxygen to the blood and removes the carbon dioxide resulting from metabolic processes. Oxygenators usually have hollow fibers past which blood flows on the outside, while oxygen, air or other gases flow through the inside of the fibers. Owing to a concentration gradient, exchange of gases, in particular of oxygen and carbon dioxide, occurs between gas and blood at the membrane. To maintain the organism, the blood is enriched with oxygen and freed of carbon dioxide. Targeted adjustment of the pH of the blood is possible in this way by changing the O₂ supply or by use of a CO₂ supply through the oxygenator.

According to yet another aspect of the invention, the gas exchange module is employed to adjust a physiological pH of the blood.

This embodiment of the use according to the invention is advantageously employed, as already mentioned hereinbefore, for example for patients suffering from acidosis or alkalosis, whether chronic or acute acidoses/alkaloses. The pH in these patients is increased (alkalosis) or reduced (acidosis), whereby cell functions and thus also organ or tissue functions may be impaired. It is possible through the use of the gas exchange module to supply gas in a targeted manner to the patient's blood, thus readjusting the pH of the blood to physiological values, i.e. to values at which the cells/organs/tissues operate as in the healthy person. As mentioned hereinbefore, the pH of arterial blood of a healthy person is generally between 7.36 and 7.45. There are various possible causes of acidosis, such as, for example, impairments of gas exchange associated with pulmonary disorders, disorders of the brain, or else be caused by metabolic impairments, such as, for example, renal failure, burns, shock, hereditary diseases. An elevated pH may occur for example when the hormone balance is impaired.

According to another aspect of the invention, the gas exchange module is employed to adjust a non-physiological pH of the blood.

This embodiment has the advantage that, for example in patients who are treated with drugs whose activity optimum is in a non-physiological pH, the pH can be guided in a targeted and controllable manner into the acidic or basic range depending on the activity optimum of the drug employed.

According to this aspect, the gas exchange module can be employed to adjust the blood to a pH of between 2.5 and 9.

The gas to be employed, or the nature of the gas, can be adapted to the particular application or to the particular use. It is particularly preferred to use a CO₂, O₂ and/or N₂ supply to the blood with the gas exchange module.

Carbon dioxide is physically dissolved in blood as carbonic acid (HCO₃⁻), the latter being in dissociation equilibrium with CO₂. The pH in the blood is altered through this dissociation equilibrium and, for example, reduced with an increased CO₂ supply, whereby it is possible to create optimal conditions for the patient or organ which is to be treated in each case by an adjustment of the pH. For example a targeted reduction of the pH is possible by supplying CO₂ if the pH of the blood was previously physiological. If the pH of the blood to be treated tends to be non-physiologically basic before the treatment, the pH can be reduced to a physiological value by supplying CO₂. Conversely, for example, the pH for example can be diverted from a non-physiological acidic pH with a supply of O₂. Also, an increase in the pH can be achieved with a supply of N₂, so that a non-physiologically acidic pH can for example be increased to a physiological pH, or a physiological pH to a non-physiological basic pH.

The invention therefore also relates to a method for adjusting a pH of blood in a patient and/or of blood in an isolated organ, where the method includes the step of employing a gas exchange module. It is particularly preferred in this connection for the organ to be perfused in isolation in a patient, and for the gas exchange module to include at least one gassing membrane, in particular a hollow fiber membrane or a flat membrane.

It is further preferred in the method for the gas exchange module to be an artificial lung or an oxygenator.

The gas exchange module is employed in the method of the invention to adjust a physiological pH of the blood, and is employed in particular when the intention is to treat patients suffering from chronic or acute acidosis or alkalosis. On the other hand, the method can also be employed to adjust a non-physiological pH of the blood if, for instance, patients are treated with drugs whose activity optimum is in the acidic or in the basic region. The method of the invention or the use according to the invention can further be employed if the pH is to be raised or lowered in patients receiving a drug therapy at a physiological pH, in order thus to inactivate, eliminate or release the drug.

The novel use of the gas exchange modules provides a simple and efficient means with which it is possible to adjust the pH of a patient's blood and/or of a target area/organ perfused in isolation, in a rapid, targeted manner and without exposing the body to additional substances.

Further advantages are evident from the description and the examples.

### BRIEF DESCRIPTION OF THE DRAWINGS

An exemplary embodiment of the invention is explained in more detail in the following description with reference to the appended drawing. This shows in

FIG. 1 diagrammatic representation of the dependence of the pH on the CO₂ partial pressure and

FIG. 2 a diagram depicting the results of an experiment on the correlation between CO₂ supply and pH.

### DETAILED DESCRIPTION OF PREFERRED EMBODIMENTS

### Example

### Conditioning of 200 ml of Blood to pH 7.3

The following design of experiment was used for this experiment: the gas exchange module employed was a hollow fiber module of the applicant (ref.: HF-PMP-90/200 Lot 2006_{—}006). This took the form of a module with about 25 polymethylpentene hollow fibers having a length of 10 cm.

The module was provided with a gas supply and with a gas outlet and put into a glass beaker. 200 ml of blood which, before the treatment, had a pH of 7.35, an oxygen partial pressure pO₂ of 40.8 and a carbon dioxide partial pressure pCO₂ of 48.6 was then used. The blood was put into the glass beaker into which the hollow fiber module had been introduced, and covered the latter completely.

Subsequently, CO₂ was supplied to the hollow fiber module, specifically at 0.1 l/min, in order to adjust the blood to a pH of <7. The CO₂ supply was then switched off, and an O₂ supply of 0.2 l/min or 0.5 l/min was adjusted to achieve maximum O₂ saturation. The O₂ gassing was then switched off.

An N₂ gassing was adjusted to 0.5 l/min in order to adjust the pH of the blood to pH>7 and in order to reach a minimal CO₂/O₂ saturation.

Samples were taken every 10 min, and the samples were immediately subjected to gas analysis.

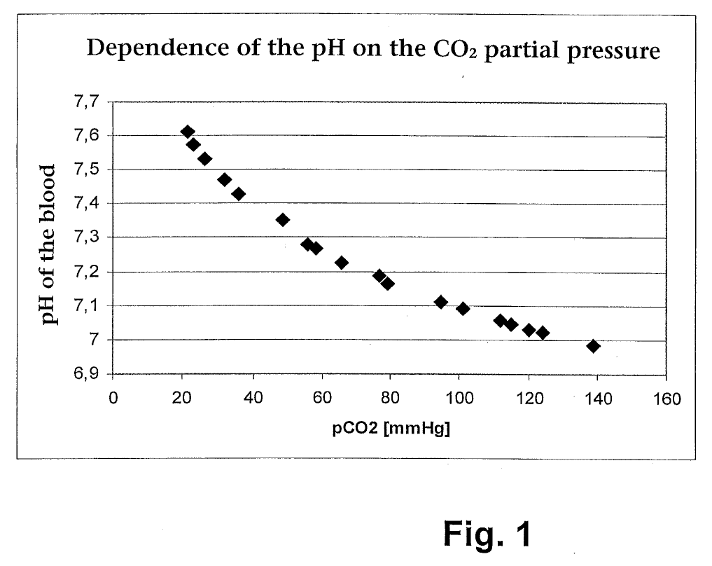

FIG. 1 shows how the pH of the blood depends on the CO₂ partial pressure pCO₂. It is evident that the pH of blood increases as the CO₂ partial pressure increases, so that the pH can be adjusted appropriately. The pH is about 7.6 at a pCO₂ of 20 mm Hg, and the pH is about 7 at a pCO₂ of 140 mm Hg.

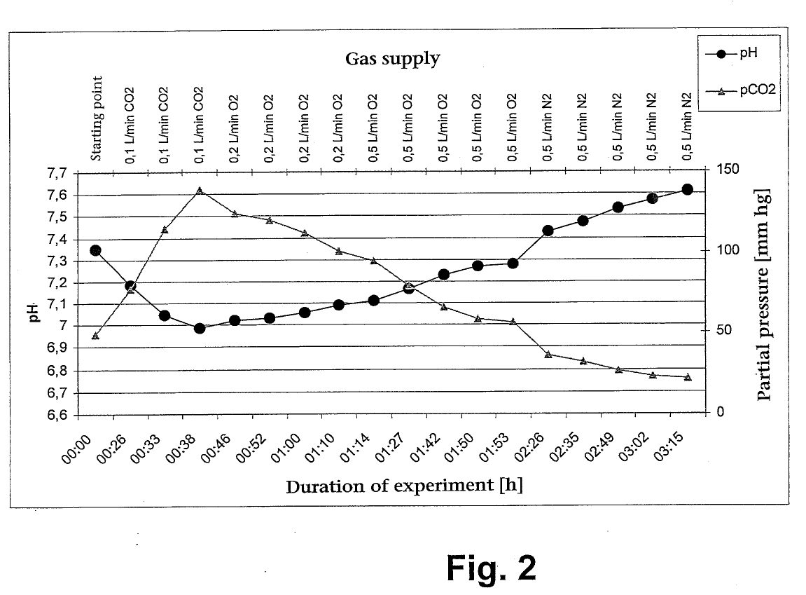

The results of the experiment described above are also shown in the diagram in FIG. 2, in which the two curves show on the one hand the change in pH (black circles) and on the other hand the change in the partial pressure pCO₂ (gray triangles). The pH was plotted against the duration of the experiment and the gas supply of the three gases.

As is evident from FIG. 1, it was possible to reduce the pH of blood below 7 by supplying CO₂ via the hollow fiber module (see measurements after 26 min, 33 min, 38 min). It was possible in turn to raise the pH by supplying O₂, in particular more quickly with a larger volume O₂ supply (see the measurements after 46 min, 52 min, 1 h, 1 h 10 min, 1 h 14 min, 1 h 27 min, 1 h 42 min, 1 h 50 min, 1 h 53 min), in particular up to the maximum CO₂/O₂ saturation of blood. It was then possible by subsequent N₂ supply to raise the pH above 7.6 (see the measurements after 2 h 26 min, 2 h 35 min, 2 h 49 min, 3 h O₂ min and 3 h 15 min).

These results show that it is possible by employing gas exchange modules to influence in a targeted way the pH of a patient's blood and also the pH of the blood in a target area/organ perfused in isolation, by supplying gases, in particular as a function of the gas used. The pH can moreover, for example, be precalculated accurately via the volumetric amount of gas.

Therefore, what is claimed, is:

1. A method for adjusting a pH value of blood, comprising the steps
establishing a connection between said blood and a gas exchange module, and
supplying gas via the gas exchange module to the blood.

2. The method as claimed in claim 1, wherein said blood is blood in a patient.

3. The method as claimed in claim 1, wherein said blood is blood in an isolated organ.

4. The method as claimed in claim 1, wherein said blood is blood in an isolated organ, and wherein the organ is perfused in isolation in a patient.

5. The method as claimed in claim 1, wherein the gas exchange module includes at least one hollow fiber membrane.

6. The method as claimed in claim 1, wherein the gas exchange module is an artificial lung.

7. The method as claimed in claim 1, wherein the gas exchange module is employed to adjust a physiological pH of the blood.

8. The method as claimed in claim 1, wherein the gas exchange module is employed to adjust a non-physiological pH of the blood.

9. The method as claimed in claim 1, wherein the gas exchange module is employed to adjust the blood to a pH of between 2.5 and 9.

10. The method as claimed in claim 1, wherein the gas is CO₂, O₂ and/or N₂.

11. A method for adjusting a pH value of blood, wherein the method includes the step of employing a gas exchange module.

12. The method of claim 11, wherein said blood is blood in a patient.

13. The method of claim 11, wherein said blood is blood in an isolated organ.
